(19) United States
(12) Patent Application Publication
(10) Pub. No.: US 2006/0130830 A1
(43) Pub. Date: Jun. 22, 2006

(54) Intra-bronchial implants for improved attachment

(75) Inventor: Robert L. Barry, Kirkland, WA (US)

Correspondence Address: Robert L. Barry;Suite 204, 2028 5th Avenue, Seattle, WA, 98121, US

(73) Assignee: Uptake Medical Corporation, Seattle, WA (US)

(21) Appl. No.: 11/222,118

(22) Filed: Sep. 7, 2005

Related U.S. Application Data
(63) Continuation-in-part of application No. 11/208,396, filed on Aug. 20, 2005.
(60) Provisional application No. 60/607,527, filed on Sep. 7, 2004.
(60) Provisional application No. 60/607,623, filed on Sep. 8, 2004.

(51) Int. Cl. A61M 16/00

(52) U.S. Cl. 128/200.26; 128/207.14; 128/207.15

(57) ABSTRACT

An obstructive device adapted to be implanted into a patient's airway comprising: at least one tissue contacting surface adapted to delivery energy to an airway wall in order to induce a fibrotic response between the device and the patient.

### CROSS-REFERENCE

This application is a continuation-in-part application of Ser. No. 11/208,396, filed Aug. 20, 2005, which is incorporated herein by reference in its entirety and to which application we claim priority under 35 USC § 120.

This application further claims the benefit of U.S. Provisional Application No. 60/607,527, filed Sep. 7, 2005 and U.S. Provisional Application No. 60/607,623, filed Sep. 8, 2005, which are incorporated herein by reference in their entirety.

### FIELD OF THE INVENTION

The present invention is related to the medical devices, systems, methods and kits for achieving lung volume reduction in a targeted region of a patient's lung,

### BACKGROUND OF THE INVENTION

Emphysema is a debilitating disease. A subtype of chronic obstructive pulmonary disease (COPD), emphysema is characterized by the destruction of the lung parenchyma, which leads to the primary pathology of emphysema, namely the dilatation and destruction of respiratory bronchioles, subsequent gas exchange abnormalities and eventual pulmonary hypertension and right heart failure as the disease progresses.

Lung volume reduction surgery (LVRS) is used to remove damaged lung tissue and is a treatment for patients with emphysema as well as other lung disorders. In this surgical procedure, about 20-30% of a patient's total lung volume is excised. While several clinical studies have shown the effectiveness of LVRS, this surgical procedure is fairly expensive and the risks of early postoperative mortality and morbidity are high in patients who are compromised by lung disease.

Recently, non-surgical, bronchoscopic approaches for achieving lung volume reduction have been proposed. In these approaches bronchoscopic lung volume reduction is achieved by implanting endobronchial sealants, plugs and valves into one or more patient airways to isolate a diseased region of a patient's lung from airflow in order to reduce a volume of a diseased lung region. Over time, the treated lung is expected to deflate or become atelectatic.

However, as with many types of medical implants, effective attachment of the device into the surrounding tissue, however, is not always readily achieved and migration of the medical implant and tissue erosion caused by the implant can be a problem. As will be recognized by those skilled in the art, the clinical performance of numerous medical devices depends upon the device being effectively anchored into the surrounding tissue. As a result of poor attachment, the implants can have a tendency to migrate. The extent to which a particular type of medical implant can move or migrate after implantation depends on a variety of factors including the type and design of the device, the material(s) from which the device is formed, the mechanical attributes (e.g., flexibility and ability to conform to the surrounding geometry at the implantation site), the surface properties, and the porosity of the device or device surface. The tendency of a device to loosen after implantation also depends on the type of tissue and the geometry at the treatment site, where the ability of the tissue to conform around the device generally can help to secure the device in the implantation site. Device migration can result in device failure and, depending on the type and location of the device, can lead to migration and/or damage to the surround tissues.

The present invention is directed to providing methods and devices for increasing the effective implantation and/or attachment of a bronchial implant inside a patient's airway.

### SUMMARY OF THE INVENTION

In the present invention, methods and device modifications are provided to secure an implantable intra-bronchial device in place in a patient's airway.

In one aspect of the invention, a bronchial implant is adapted to be anchored within an airway. As is further described anchoring of the implant can be immediate and/or gradual and can be achieve via the application of energy (RF, hot air, hot liquid, vapor, laser, microwave, high intensity ultrasound, cryo-energy) which induces immediate adhesion of the implant and/or gradual adhesion, with eventual fibrosis in the surround airway tissue facilitating anchoring of the bronchial device/implant in situ.

Within various embodiments, fibrosis can be induced in a variety of ways. For example, in addition to causing immediate attachment of an implant, the application of energy can induce fibrosis. Alternatively or in conjunction, fibrosis can be induced via the local release of specific fibrosing or irritant agents, such as talcum powder, metallic beryllium and oxides thereof, copper, silk, silica, crystalline silicates, talc, quartz dust, and ethanol; a component of extracellular matrix selected from fibronectin, collagen, fibrin, or fibrinogen; a polymer is selected from the group consisting of polylysine, poly(ethylene-co-vinylacetate), chitosan, N-carboxybutylchitosan, and RGD proteins; vinyl chloride or a polymer of vinyl chloride; an adhesive selected from the group consisting of cyanoacrylates and crosslinked poly(ethylene glycol)-methylated collagen; an inflammatory cytokine (e.g., TGF.beta., PDGF, VEGF, bFGF, TNF.alpha., NGF, GM-CSF, IGF-a, IL-1, IL-1-.beta., IL-8, IL-6, and growth hormone); connective tissue growth factor (CTGF); a bone morphogenic protein (BMP) (e.g., BMP-2, BMP-3, BMP-4, BMP-5, BMP-6, or BMP-7); leptin, and bleomycin or an analogues or derivative thereof. Optionally, an intrabronchial device may additionally comprise a proliferative agent that stimulates cellular proliferation. Examples of proliferative agents include: dexamethasone, isotretinoin (13-cis retinoic acid), 17-.beta.-estradiol, estradiol, 1-a-25 dihydroxyvitamin D.sub.3, diethylstibesterol, cyclosporine A, L-NAME, all-trans retinoic acid (ATRA), and analogues and derivatives thereof.

In one embodiment, the fibrosing agent may be associated with the implant prior to the implant being placed within the animal. For example, the agent (or composition comprising the agent) may be coated onto an implant, and the resulting device then placed within the animal. In addition, or alternatively, the agent may be independently placed within the animal in the vicinity of where the device is to be, or is being, placed within the animal. For example, the agent may be sprayed or otherwise placed onto the tissue that can be contacting the medical implant or may otherwise undergo scarring.

In yet another aspect of the invention, the intra-bronchial implants are further anchored mechanically to the biological tissue of an airway. For example, implants can be anchored to the surrounding tissues by physical and mechanical means (e.g., screws, flanges, or lips) or by friction in conjunction with the application of energy and/or fibrosing agents to further affix the implant in place.

In yet another aspect of the invention, attachment of the implant can be facilitated by mechanically altering the surface characteristics of the device. For example, tissue contracting surfaces of an implant can be scored or abraded so that the roughened surfaces promote cell and tissue adhesion for better affixing an intra-bronchial implant in a patient's airway. Implants with altered surface characteristics can be employed alone or in conjunction with the application of energy and/or fibrosing agents to further affix the implant in place.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an anterior view of a pair of human lungs and trachea;

FIG. 2 is an anterior view of the trachea and bronchial tree;

FIG. 3 is a schematic illustration of one embodiment of an intra-bronchial implant in accordance with one embodiment of the present invention; and

FIG. 4 is a schematic illustration of one embodiment of an intra-bronchial implant in accordance with one embodiment of the present invention.

### DETAILED DESCRIPTION OF THE DRAWINGS

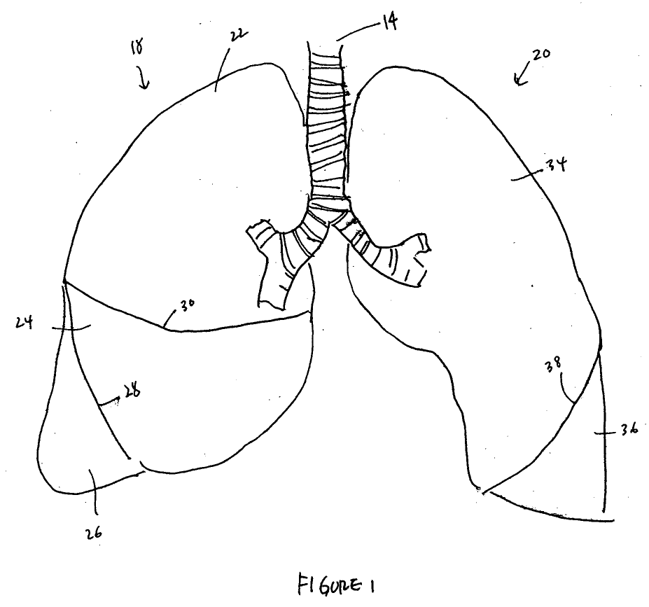

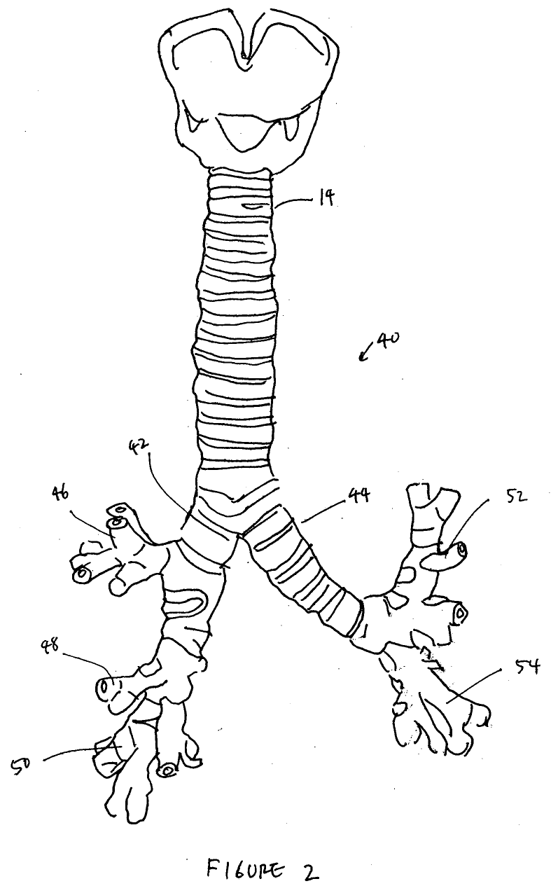

FIG. 1 shows an anterior view of a pair of human lungs, the trachea 14 and a bronchial tree 16 that provides a ventilation pathway into and out of the lungs. For clarity of illustration, FIG. 1 shows only a portion of the bronchial tree 16, which is described in more detail below with reference to FIG. 2.

The lungs include a right lung 18 and a left lung 20. The right lung 18 includes three lobes, the right upper lobe 22, the right middle lobe 24, and the right lower lobe 26. The lobes 22, 24, 26 are separated by two interlobar fissures, including a right oblique fissure 28 and a right transverse fissure 30. The right oblique fissure 28 separates the right lower lobe 26 from the right upper lobe 22 and from the right middle lobe 24. The right transverse fissure 30 separates the right upper lobe 22 from the right middle lobe 24.

The left lung 20 includes lung regions comprised of two lobes, including the left upper lobe 34 and the left lower lobe 36. An interlobar fissure comprised of a left oblique fissure 38 of the left lung 32 separates the left upper lobe 34 from the left lower lobe 36. The lobes 22, 24, 26, 34, 36 are directly supplied air via respective lobar bronchi, as described in detail below with reference to FIG. 2.

FIG. 2 shows an anterior view of the trachea 14 and a portion of the bronchial tree 40, which includes a network of bronchial passageways, as described below. The trachea 14 divides at a distal end into two bronchial passageways comprised of primary bronchi, including a right primary bronchus 42 that provides direct air flow to the right lung 18, and a left primary bronchus 44 that provides direct air flow to the left lung 20. Each primary bronchus 42, 44 further divide into a plurality of lobar bronchi. The right primary bronchus 42 divides into a right upper lobar bronchus 46, a right middle lobar bronchus 48, and a right lower lobar bronchus 50. The left primary bronchus 44 divides into a left upper lobar bronchus 52 and a left lower lobar bronchus 54. Each lobar bronchus, 46, 48, 50, 52, 54 directly feeds fluid to a respective lung lobe, as indicated by the respective names of the lobar bronchi. The lobar bronchi yet again further device into segmental bronchi, which provide air flow to the bronchopulmonary segments discussed above. The diameter of the internal lumen for a specific bronchial passageway can vary based on the bronchial passageway's location in the bronchial tree (such as whether the bronchial passageway is a lobar bronchus or a segmental bronchus) and can also vary from patient to patient. However, the internal diameter of a bronchial passageway is generally in the range of 3 millimeters (mm) to 10 mm, although the internal diameter of a bronchial passageway can be outside of this range. For example, a bronchial passageway can have an internal diameter of well below 1 mm at locations deep within the lung.

The bronchial passageway defines a pathway through which air, fluids, etc. can flow to and from a lung. In addition, the lungs may be characterized as a mass exchanger in which oxygen is delivered via the bronchial passageways through the alveoli to blood and carbon dioxide is removed from the blood for exhalation. The efficiency of the lungs, in terms of the exchange of gaseous materials at the blood/gas interface, is dependent in-part on the ventilation of each lung. The term “ventilation” refers to the movement of or the exchange of oxygen-rich air from outside the patient's body into the lung where the air is mixed with relatively oxygen deficient air through the course of breathing. The ventilation function of a patient's lungs can be determined and monitored by measuring the resistance and compliance of the airways of the lung. The resistance and compliance within different regions of the lungs affect the distribution of pulmonary ventilation. “Resistance” refers to the flow resistance due to an obstruction or a restriction within a respiratory passageway to the passage or flow of a gas to and from the lungs. “Compliance” refers to the flexibility or elasticity of the lungs as they expand and contract during a respiratory cycle. In patients with emphysema and other lung diseases, the patient's ventilation may be compromised due to altered resistance and compliance characteristics of the lungs.

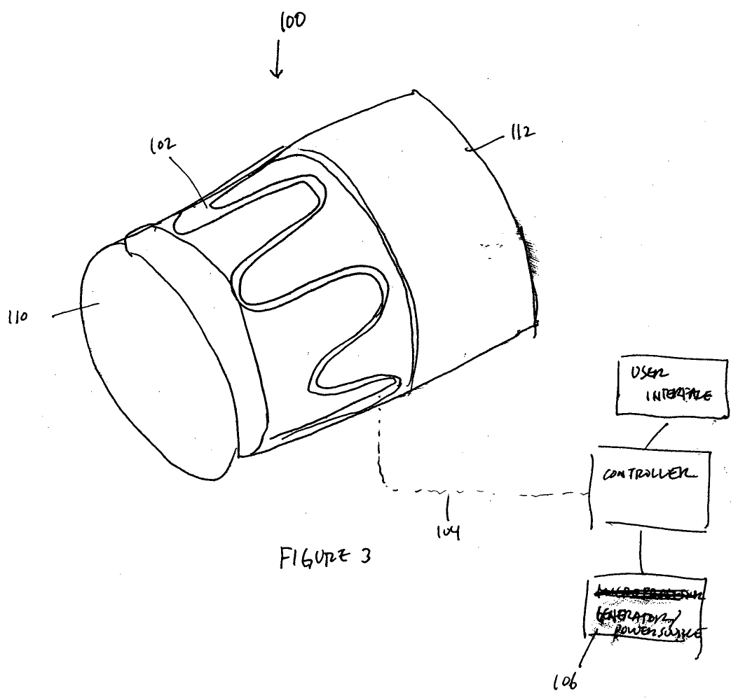

FIG. 3 shows a schematic illustration of an intra-bronchial implant 100 in accordance with one embodiment of the present invention. In this embodiment, the implant 100 includes one more energy delivery surfaces 102 disposed on a tissue contacting surface of the implant for thermally attaching the implant inside a patient's airway. In one embodiment, energy delivery surfaces is one or more RF electrodes 102 that are functionally connected via one or more electrical connections 104 to an RF generator 106 for energizing electrodes, an ultrasound transducer, an optic fiber for laser or infrared transmission, or other elements for electromagnetic transmission of energy to the surfaces of a patient's airway to promote immediate attachment of the implant inside the airway upon activation of the energy delivery surface. As will be recognized by those skilled in the art, application of energy (sufficient to raise native tissue temperature above about 45° C.) will heat and melt tissue collagen to create a type of biologically molten glue. In addition, the delivery of energy will also cause a tissue injury response wherein a biological healing or repair response is induced. The repair of tissues following an injury (including a thermal injury) generally involves: (1) regeneration (the replacement of injured cells by cells of the same type) and (2) fibrosis (the replacement of injured cells by connective tissue). There are four general components to the process of fibrosis (or scarring) including: formation of new blood vessels (angiogenesis), migration and proliferation of fibroblasts, deposition of extracellular matrix (ECM), and remodeling (maturation and organization of the fibrous tissue). This injury response (including fibrosis) will form scar/fibrotic tissues in and around the implant further attaching the implant inside the airway.

As is further shown in FIG. 3, implant further includes a distal end 110 and a proximal end 112 and has a generally cylindrical shape. However, the implant can be configured in different shapes, sizes and include one or more functional structures adapted for delivering, securing and detaching the implant into any bronchial (main, segmental or sub-segmental) passageway. For example, at the proximal end, the implant can include a detachment mechanism that can be used to release the implant from a delivery mechanism once the implant has been attached to tissues at a desired location.

As will be readily understood by those skilled in the art, more efficient, immediate attachment of the device inside the airway can be promoted by ensuring good fit and contact between the energy delivering surfaces (i.e. RF electrodes, etc) of the implants and the tissues. To this end, implant can be adapted to include a flanges, struts or other mechanical structures adapted to grip the interior walls of the airway to ensure tissue to energy delivery surface contact and to prevent migration or movement of the implant during the implantation procedure. In yet another embodiment, the implant can be adapted to include one or more suction ports that allow a suction to be pulled so that the surrounding tissues are vacuum-pressed to the implant and energy delivery surfaces for efficient energy transfer.

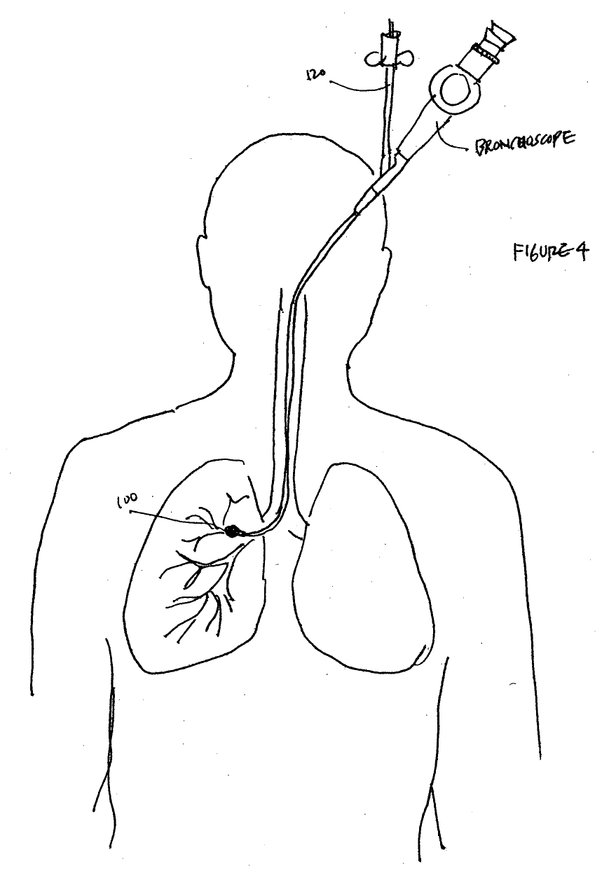

FIG. 4 shows another embodiment of the invention, wherein the implant is a releasable and compliant inflation member or balloon releasable from the distal end of a bronchoscopically deliverable treatment catheter 120. In this embodiment, the implant 100 is manufactured from a deformable material, such as conductive materials such as silicone or a deformable elastomer or the like, which is inflatable with a hot or cryo-liquid (water, or saline), air (oxygen, inert noble gas, carbon dioxide, etc) or vapor (water, saline or the like). In this embodiment, the hot or cold air, vapor or liquid transfers energy to the tissues of the airway and facilities its immediate attachment. In addition, it this process also initiates a scarring/healing response, which serves to further attach the implant inside the airway.

In yet another embodiment, the various implants of the present invention can be further adapted to include or deliver one ore more fibrosing agents to promote the scarring/healing process. For example, agents, such as talcum powder, metallic beryllium and oxides thereof, copper, silk, silica, crystalline silicates, talc, quartz dust, and ethanol; a component of extracellular matrix selected from fibronectin, collagen, fibrin, or fibrinogen; a polymer is selected from the group consisting of polylysine, poly(ethylene-co-vinylacetate), chitosan, N-carboxybutylchitosan, and RGD proteins; vinyl chloride or a polymer of vinyl chloride; an adhesive selected from the group consisting of cyanoacrylates and crosslinked poly(ethylene glycol)-methylated collagen; an inflammatory cytokine (e.g., TGF.beta., PDGF, VEGF, bFGF, TNF.alpha., NGF, GM-CSF, IGF-a, IL-1, IL-1-.beta., IL-8, IL-6, and growth hormone); connective tissue growth factor (CTGF); leptin, and bleomycin or an analogues or derivative thereof can be disposed on the implant. Optionally, an intrabronchial device may additionally comprise a proliferative agent that stimulates cellular proliferation. Examples of proliferative agents include: dexamethasone, isotretinoin (13-cis retinoic acid), 17-.beta.-estradiol, estradiol, 1-a-25 dihydroxyvitamin D.sub.3, diethylstibesterol, cyclosporine A, L-NAME, all-trans retinoic acid (ATRA), and analogues and derivatives thereof. In one example, intrabronchial implants such as those described in U.S. patent application Ser. No. 11/092,123, entitled “Bronchial Flow Control Devices and Method of Use, may including one more fibrosing agents on a tissue contacting surface of the implant to promote attachment of the implant inside an airway.

In yet another embodiment of the invention, the various implants of the invention can be adapted as a one way valve (for example as further described in Ser. No. 11/092,123). In this embodiment of the invention, the implant comprises one or more energy delivery surface and is operationally coupled to a vacuum pump that can be used to draw a vacuum in an airway before or after the implant has been attached inside a patient's airway. As will be recognized by those skilled in the art, the application of a low vacuum will facilitate the collapse of the desired tissue region. In yet another embodiment, the implant may further comprise a removable inner portion consisting of a valve or pint, which an be removed to allow any trapped air from an obstructed airway to diffuse past the airway and out past the obstructions.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed:

1. An obstructive device adapted to be implanted into a patient's airway comprising: at least one tissue contacting surface adapted to delivery energy to an airway wall in order to induce a fibrotic response between the device and the patient.

2. An obstructive device adapted to be implanted into a patient's airway comprising: at least one tissue contacting surface comprising at least one energy delivery member and a fibrosing agent where the fibrosing agent induces a fibrotic response between the device and the patient in which the device is implanted.

3. A method for inducing the partial or total collapse of a targeted region of a patient's lung comprising: advancing an obstructive device into a patient's airway and thermally fixing the obstructive device inside the airway by applying energy to a interface between the airway and a tissue contacting surface of the device.
